(19) United States
(12) Patent Application Publication
(10) Pub. No.: US 2018/0078328 A1
(43) Pub. Date: Mar. 22, 2018

(54) PRE-SURGICAL PULMONARY NODULE LOCALIZATION SYSTEMS AND METHODS

(71) Applicant: Regents of the University of Minnesota, Minneapolis, MN (US)

(72) Inventors: Michael Rosenberg, Eagan, MN (US); Sean Lester Moen, Saint Paul, MN (US); Jafar Golzarian, Plymouth, MN (US)

(21) Appl. No.: 15/705,403

(22) Filed: Sep. 15, 2017

Related U.S. Application Data
(60) Provisional application No. 62/395,454, filed on Sep. 16, 2016.

(51) Int. Cl. A61B 90/00; A61B 34/20; A61B 17/04; A61B 17/34

(52) U.S. Cl. CPC A61B 90/39; A61B 34/20; A61B 17/0469; A61B 17/3403; A61B 2017/3413; A61B 2090/3991; A61B 2090/3762; A61B 2090/3908; A61B 2090/3983; A61B 2090/3987; A61B 2034/2063

(57) ABSTRACT

A nodule localization system includes a localization structure. The localization structure is arranged to extend from a nodule to a suture, and the entire coil can be located in the lung such that the suture extends through the pleural space. During normal respiration or other movement, the suture will move to be positioned mostly or entirely in the pleural space such that a surgeon can follow the suture to the coil and resect the nodule.

### RELATED APPLICATION

The present application claims the benefit of U.S. Provisional Application No. 62/395,454 filed Sep. 16, 2016, which is hereby incorporated herein in its entirety by reference.

### TECHNICAL FIELD

Embodiments relate to surgical instruments, systems, devices, and/or methods for locating and tracking the position of a nodule.

### BACKGROUND

To surgically remove a nodule or other object, an imaging system can be used to identify the exact location of that object, and a localization wire is inserted into the patient and placed into the nodule. Subsequently, a surgeon can follow the localization wire to the object, and remove it. This process is sometimes referred to as tumor localization, wire localization, pre-surgical nodule localization, or computer aided tomography wire localization.

Between the time that the localization wire is placed and the time that the surgeon removes the object, the localization wire can become dislodged. In the case of pulmonary nodule localization, this can occur because the patient's respiration cycle causes relative movement between the object and the patient's skin, where the localization wire is conventionally affixed.

One conventional solution to this problem is to use a hook-shaped wire, such that traction applied on the wire by respiration is less likely to cause the wire to dislodge from the nodule or other device. However, the proximal (more superficial portion of the hook wire is affixed to the skin which has still resulted in dislodgment or migration of the distal hook portion away form the localization site. This migration can result in trauma to the lung and potential difficulties precisely locating the nodule intraoperatively. Patients can often wait for long periods, up to 12 hours after localization wires are placed, due to surgical suite or CT room scheduling constraints. Some short hook systems have been shown to reduce dislodgement of the localization wire in some patients. Shortcomings of such short hook systems include unsuccessful placement caused by too shallow a puncture with the introducer needle, and some instances of hemorrhages into the lung or pleural space.

In some systems, a coil is used to allow for relative movement of the skin and the nodule or other object. Such a coil is deployed partly in or adjacent to the nodule/surgical target (distal end of coil) and the remainder (proximal end) is positioned in the pleural space, a small potential space between the two layers of the pleura. The pleura are thin coverings that protect and cushion the lungs, positioned between the lungs and chest cavity. Failure to place the coil in the pleural region can result in dislodgement of the distal end of the coil and loss of localization site for the surgeon and potential damage to the soft tissue surrounding the delivery tract, causing injury or hemorrhaging in the patient.

The intra-pleural portion essentially floats within the pleural space preventing traction on the portion of the coil within the lung and potential coil dislodgement. If the portion of the coil that is intended to lie within the pleural space is inadvertently deployed in the more superficial soft tissue tract, the intra-pulmonary portion of the coil will then retract with respiratory motion due to the proximal portion being fixed in place by the superficial soft tissues, thereby resulting in dislodgment from the desired localization site. It is very challenging to precisely place the proximal end within the pleural space as this is often only a collapsed potential space that is not easily localized. In addition to loss of localization site for the surgeon, coil dislodgement can result in injury or hemorrhaging from the lung or tract.

### SUMMARY

According to embodiments herein, a localization system is described that is not displaced during normal respiration of the patient prior to surgery, and also a system that does not cause injury to the patient by introducing large or complex structures such as hooks or barbs at the site of the nodule.

Embodiments relate to localization structures or systems having elastic, dynamic properties that prevent dislocation prior to surgery. In embodiments, a suture can be attached to a spring or coil, such that the localization system has a coil portion that fixes itself within the desired position and a suture portion that traverses the pleura and tract to the skin site, obviating the challenge to precisely localize and deploy a device within the pleural space. The suture can be trimmed about flush with the skin surface and allowed to retract freely with respiration preventing any traction on the coil portion of the device next to the surgical target within the lung. This ability to expand, contract, and/or move with the patient's respiratory cycle reduces or eliminates the chance that the device will become dislodged, and also enables a surgeon to find the suture within the pleural space more easily.

By attaching the spring or coil to the suture, deployment time can be reduced, limiting radiation exposure and increasing accuracy of localization. Furthermore, since the spring or coil need not be positioned in the pleural space, there is a reduced potential for damage to the soft tissues of the delivery tract as compared to conventional systems. Furthermore, the embodiments described below facilitate nodule resection, allowing the surgeon to follow the easily identifiable suture down to the coil and surgical target.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Embodiments may be more completely understood in consideration of the following detailed description of various embodiments of the invention in connection with the accompanying figures, in which:

FIG. 1A is a partially cut-away plan view of a coil localization system arranged in a delivery needle according to an embodiment.

FIG. 1B is a flowchart of a method for using the coil localization system of FIG. 1A.

FIGS. 2A and 2B are schematic views of a coil localization system according to an embodiment.

FIG. 3 is a perspective view of a suture affixed to a coil at the proximal end of a deployed coil localization system according to an embodiment.

FIG. 4 is a simplified view of a human torso including a lung nodule, depicting relative movement of a lung and a remainder of the torso at a pleural space.

FIG. 5 is a prior art view of a conventional coil localization system.

FIGS. 6A-6C are simplified views of a human torso and coil localization system according to an embodiment.

FIGS. 7A-7C depict steps for positioning a suture in a pleural space according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

Embodiments discussed herein include a suture at the proximal end of a localization system and a coil or other elastic structure attached to the suture. The localization coil extends to the nodule or other object that is to be surgically removed. Respiration of the patient does not cause dislocation of the distal end of the localization coil, because the force exerted on the distal end of the coil is reduced or eliminated as the elastic structure expands or contracts to permit relative movement between the proximal and distal ends.

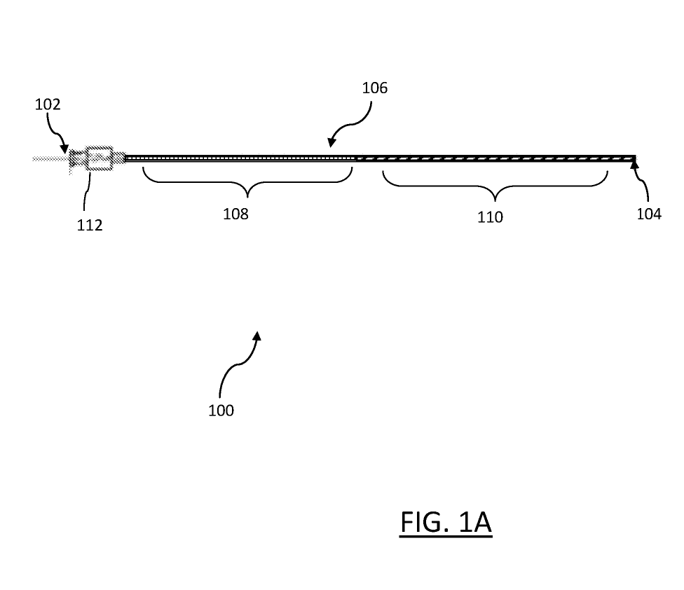

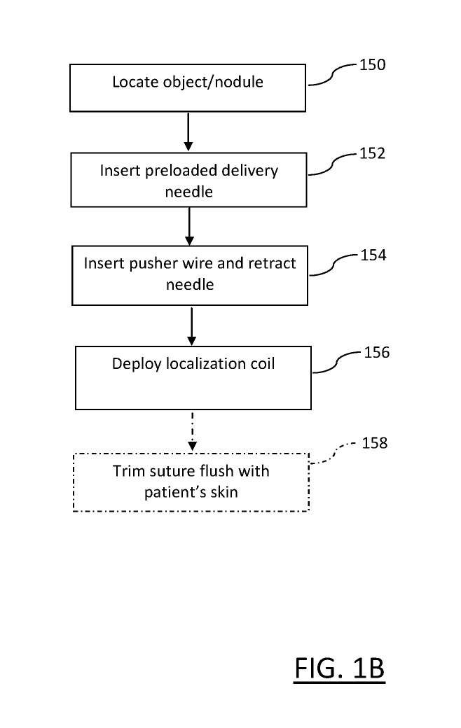

FIG. 1A is a partially cut-away view of a system 100 for delivering a localization coil and suture according to an embodiment. As shown in FIG. 1A, coil localization system 100 extends between a proximal end 102 and a distal end 104 of a deliver needle 106. Coil localization system 100 can be inserted into a patient, with proximal end 102 arranged outside the patient or at the patient's skin, while distal end 104 is arranged at the site where an object that is being removed is located, such as a nodule. This facilitates delivery of an object at distal end 104, based on manipulation of system 100 by a surgeon or other user at proximal end 102.

Arranged within delivery needle 106 are pusher wire 108 and localization structure 110. In use, distal end 104 of delivery needle 106 can be positioned in a patient during aided tomography wire localization or some other method that facilitates placement of localization structure 110 at a nodule. An operator such as a surgeon can manipulate a needle hub 112 having any of a variety of controllers, buttons, or other actuating features (not shown) at proximal end 102 to move pusher wire 108 relative to delivery needle 106. In particular, once distal end 104 is placed at the nodule, pusher wire 108 can be used to keep localization structure 110 in place while delivery needle 106 is removed from the patient.

In embodiments, localization structure 110 can be arranged substantially linearly while housed within delivery needle 106. In such embodiments, as localization structure 110 exits from distal end 104 of delivery needle 106, localization structure 110 or some portion thereof can transition from straight and linear to coiled. This permits the use of a relatively smaller delivery needle 106, as the cross-sectional profile from the distal end 104 need only be wide enough to circumscribe the thickness of the localization structure 110 in its straightened-out form, rather than its coiled form. In some embodiments, localization structure 110 can include a shape memory alloy or other material that permits selectively coiling or un-coiling. In embodiments, delivery needle 106 can be delivered to a surgeon or other operator pre-loaded with pusher wire 108 and localization structure 110.

Localization structure 110 can include both a coiling and a non-coiling portion. For example, in embodiments described herein, localization structure 110 includes a coil portion and a suture portion. In embodiments, it is desirable to position such a coil portion at the nodule. FIG. 1B is a flowchart showing a method for using a localization coil according to an embodiment.

In a first step 150, an object such as a nodule is located. The nodule or other object can be located using a scout scan using, for example, x-ray, ultrasound, tomography, or any other imaging system.

At a second step 152, a preloaded delivery needle is inserted into the patient. The delivery needle is preloaded in that it includes both a pusher wire (e.g., 108) and a localization structure (e.g., 110). The localization structure (e.g., 110 of FIG. 1A) is arranged at the location of the object or nodule identified in the scout scan of step 150.

At a third step 154, the pusher wire (e.g., pusher wire 108 of FIG. 1A) is inserted into the delivery needle. As shown in FIG. 1A, insertion of pusher wire or retraction of the delivery needle 106 causes pusher wire 108 to push localization structure 110 towards distal end 104.

At a fourth step 156 of the embodiment shown in FIG. 1B, localization coil (e.g., localization structure 110 of FIG. 1A) is deployed. Using pusher rod 108 to keep localization structure 110 in place, delivery needle 106 can be retracted, leaving localization structure 110 attached to the nodule that was previously located at first step 150. As localization structure 110 leaves delivery needle 106, it can coil up into a helical shape or some other shape which permits lengthwise expansion and contraction. In embodiments, the entire coil portion of localization structure 110 can be deposited at the nodule, and the suture attached to the coil portion can extend from the coil through the pleural portion and to the skin of the patient or beyond.

At a fifth step 158 (optional), a suture is affixed to the skin of the patient. The suture is positioned at a portion of localization coil 110 near to the proximal end 102, in embodiments. The suture thereby attaches one end of localization coil 110 to the patient's skin while the other end of localization coil is positioned at the nodule identified in first step 150. As described in more detail below, in embodiments it may be preferable to leave the suture unsecured to the skin of the patient, as this permits the suture to move to the pleural space in the chest where it can later be found by the surgeon.

After placement of a guidewire system (e.g., guidewire system 100 of FIG. 1A using the method described with respect to FIG. 1B), the patient can be taken to a surgical suite for the removal of the nodule. Often, there is a delay before the surgical suite becomes available, and this delay can be up to several hours. During this time, respiration of the patient or other movement of the patient can cause a change in length of localization structure 110. The coiled, helical, or other expansible form of localization structure 110 permits this change in length without producing excess traction on the nodule or the suture that it extends between. Alternatively, in embodiments where the coil is entirely deposited at the nodule and a suture extends therefrom, respiration can draw the suture into the pleural space.

In embodiments, localization structure 110 can have a length that is between about 8-10 cm, for example about 8.85 cm. In alternative embodiments, nodules may be located relatively closer or further from the skin of the patient, and the length of localization structure 110 can be adjusted accordingly.

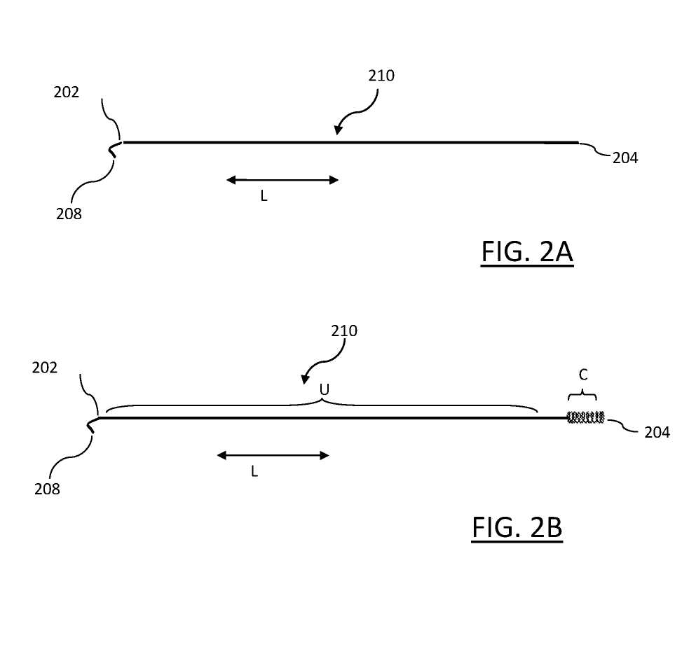

FIG. 2A is a schematic view of an embodiment of a localization structure 210 arranged along a lengthwise direction L. Localization structure 210 extends in the lengthwise direction from a proximal end 202 to a distal end 204. When loaded into a delivery needle 106 as described above with respect to FIGS. 1A and 1B, proximal end 202 and distal end 204 are arranged parallel to proximal end 102 and distal end 104 of delivery needle 106. At proximal end 202 is suture 208. Suture 208 can be attached to the skin of a patient, as described above with respect to FIG. 1B.

FIG. 2B shows localization structure 210 of FIG. 2A, in a partially deployed state. At distal end 204 is coiled portion C. Coiled portion C can be caused to coil due to shape memory properties of localization structure 210. In embodiments, localization structure 210 will coil as it exits distal end 104 of delivery needle 106. In the embodiment shown in FIG. 2B, coiled portion C is unrestrained and allowed to coil, whereas uncoiled portion U is restrained, for example by delivery needle 106. In embodiments, at least some portion of localization structure 210 can remain uncoiled portion U (such as, for example, a suture) even when localization structure 210 is fully deployed.

Various embodiments will have different widths, in addition to different lengths. In one embodiment, elastic structure 208 is a coil having a diameter of about 8 mm. In alternative embodiments, relatively larger or smaller coils could be used, or elastic structures other than coils could be used. In embodiments, elastic structure 208 is designed to be positioned on the skin of a patient, attached to the skin with a suture or other fastener. In those embodiments, the width of elastic structure 208 is of less importance, as it does not affect the size of the incision that must be made on the patient.

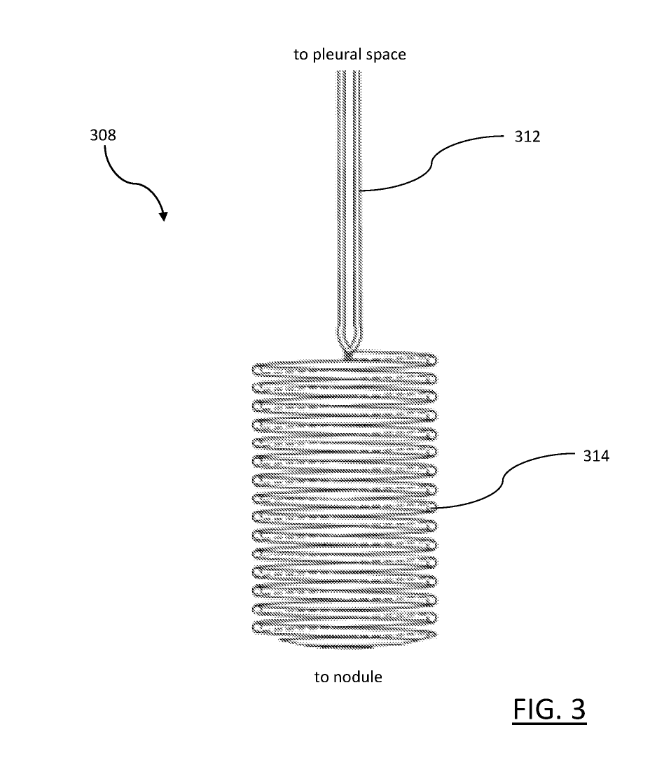

FIG. 3 depicts an elastic structure 308, according to an embodiment. In the embodiment shown in FIG. 3, coil 314 is attached to suture 312. In use, suture 312 can be left free in the needle access puncture, or alternatively suture 312 could be attached to the patient's skin. Coil 314 can be attached to a nodule in the patient's lung. During respiration or other movement of the patient, suture 312 can be pulled into the pleural space adjacent to the lung. Because coil 314 is located at the nodule and not in the pleural space, it also does not expand or contract in contact with soft tissues through which the guidewire has been routed, and therefore does not cause damage to the lining of the pleural space.

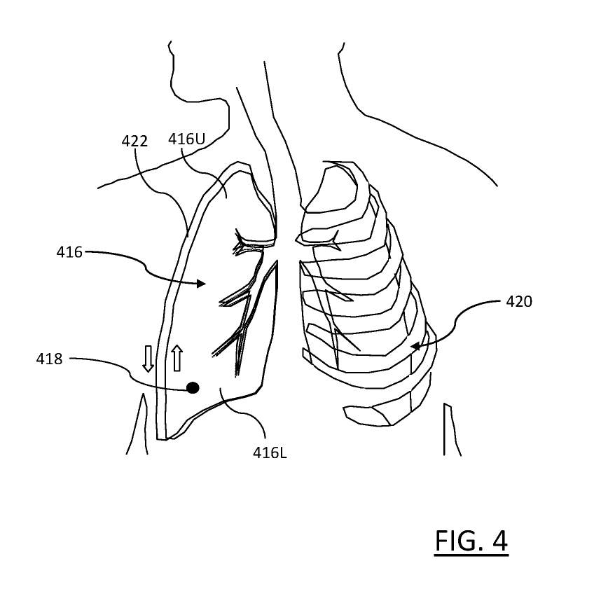

FIG. 4 is a simplified cross-sectional view of a lung 416 having a lower portion 4169L and an upper portion 416U, and including a nodule 418. Also shown in FIG. 4 are ribs 420 (removed from the left side of the Figure to more clearly depict the features of lung 416) and pleural space 422.

Pleural space 422 is positioned between lung 416 and ribs 420. Lung 416 can move relative to ribs 420 during respiration or other movement of the patient. For example, as depicted in FIG. 4, the arrows show movement of the lung upwards relative to the tissues attached to ribs 420. Such movement could occur during exhalation of the patient.

In order to provide a guidewire to a nodule, conventionally a scan is done of the patient and a needle is inserted between two of the ribs 420 and a coil is deposited. The coil attaches to the nodule and then the surgeon or other operator removes the needle until he or she has positioned the tip of the needle at the pleural space 422. The operator can then deposit the coil into the pleural space 422. For example, this can be accomplished by using a pusher rod to deposit a large quantity of coil at an angle relative to the pleural space 422 when the needle is at exactly the right position.

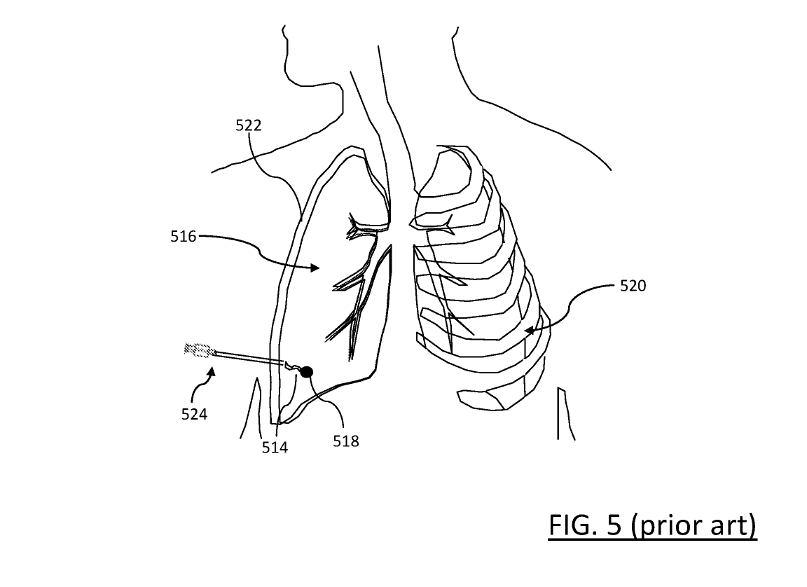

FIG. 5 shows this conventional system for depositing coil 514 attached to nodule 518 from needle 524.

Depositing a coil in this way can be difficult due to the small thickness of pleural space 522, as well as the relative movement of lung 516 relative to ribs 520 during normal respiration. Further complicating this procedure, nodules 518 positioned in the lower portion (e.g., 416L) can move relatively more with respect to ribs 520, when compared to nodules 518 positioned in the upper portion (e.g., 416U).

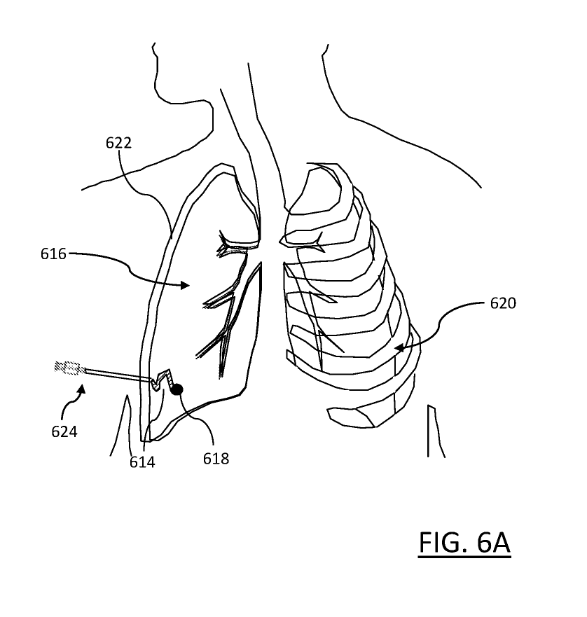

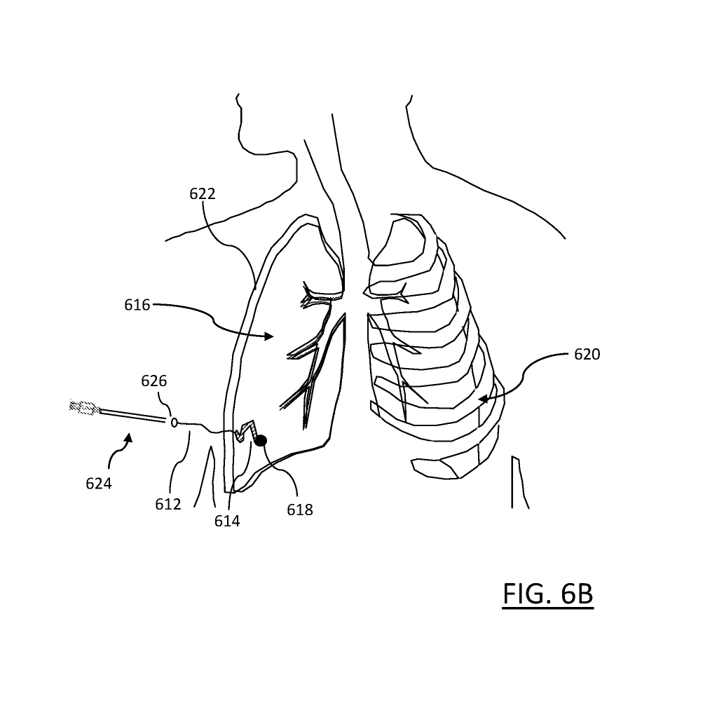

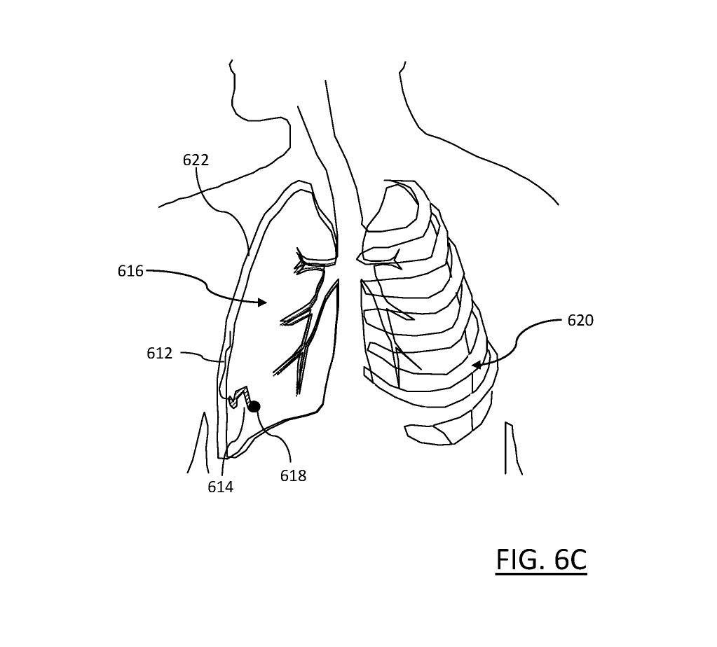

FIGS. 6A-6C depict injection of a guidewire according to an embodiment. Lung 616 includes nodule 618 that will be resected. As shown in FIG. 6A, needle 624 has deposited coil 614 at nodule 618. Coil 614 is completely deposited within lung 616, and does not extend to pleural space 622.

FIG. 6B shows needle 624 fully retracted from insertion site 626. Coil 614 and suture 612 are arranged such that the coil portion of the guidewire (614) is completely within lung 616, while the suture portion of the guidewire (612) extends from coil 612 to insertion site 624. In embodiments, a surgeon or other operator can cut suture 612 at insertion site 626. FIG. 6C shows the final state of the guidewire system before resection of nodule 618.

Due to tidal movement of lung 616 relative to ribs 620, suture 612 is pulled up and down through pleural space 622. Eventually, the entirety of suture 612 is pulled away from insertion site 626 and into pleural space 622. This is advantageous for a surgeon who later resects nodule 618, because suture 612 is easy to find in pleural space 622 and follow to nodule 618.

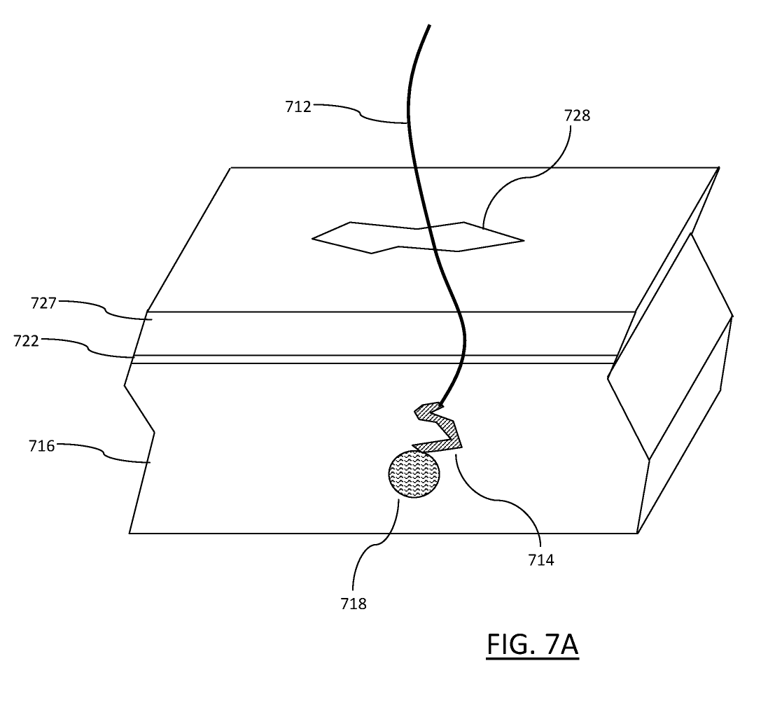

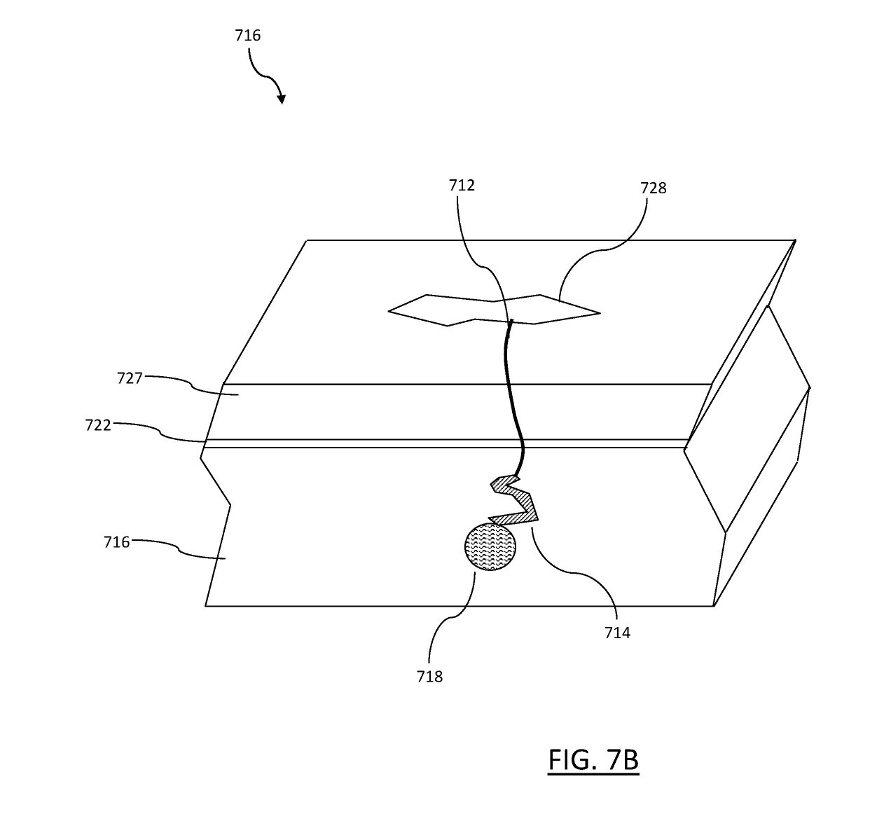

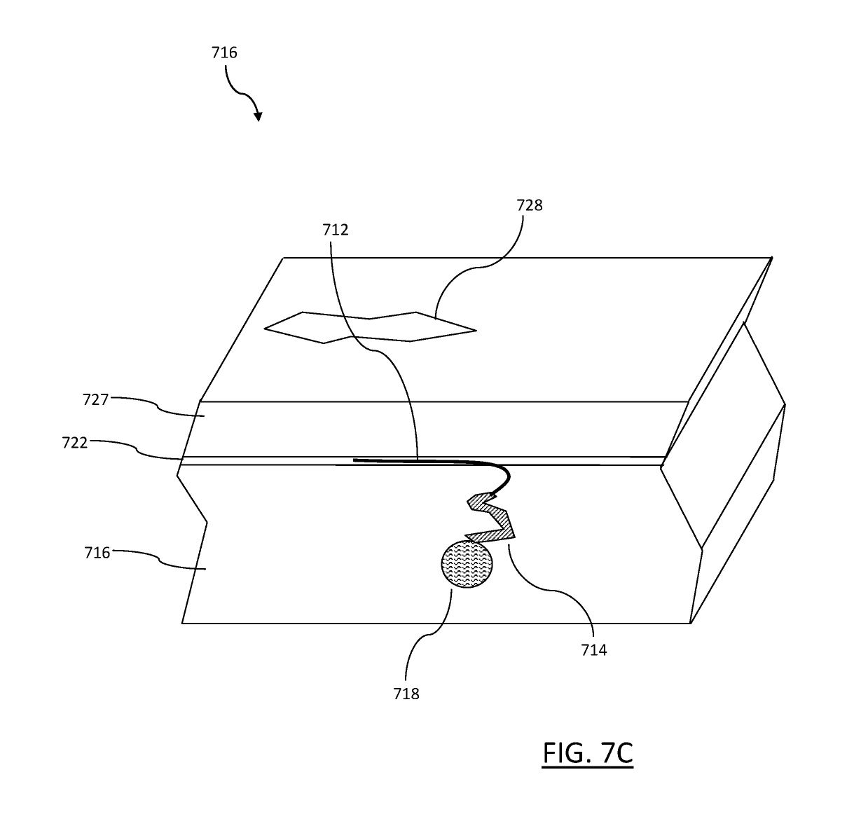

FIGS. 7A-7C depict steps that result in positioning of the suture within the pleural space according to an embodiment. Each of FIGS. 7A-7C depicts a lung 716 having a nodule 718. Lung 716 is adjacent to a pleural space 722 that separates it from the rest of the body 727. Here, body 727 is shown as a thin layer, but in embodiments body 727 could include rib bones or other obstacles that prevent easy manipulation of tools or objects within lung 716.

Incision 728 is made at a skin surface of body 727, through which a delivery needle or other mechanism can be advanced to nodule 718. Coil 714 is affixed to nodule 718 as a part of the nodule removal procedure described above. Suture 712 is attached to coil 714 as described with respect to FIG. 3, above, and extends through incision 728.

As shown in FIG. 7A, suture 712 extends through incision 728, which is an insertion site. At FIG. 7B, suture 712 has been trimmed to the surface of the skin at incision 728. In embodiments, after coil 714 is affixed to nodule 718, a medical practitioner can trim suture 712 close to the surface of the skin with scissors or other cutting objects.

Due to the movement of lung 716 due to respiration or repositioning, lung 716 and body 727 can undergo relative movement along the pleural space 722 (e.g., left-to-right or front-to-back with respect to the orientation shown in FIGS. 7A-7C). As shown in FIG. 7C, suture 712 is retracted into pleural space 722. This retraction can be caused by the relative movement of lung 716 and body 727 about pleural space 722. As depicted in FIG. 7C, body 727 has moved to the left with respect to lung 716 (as shown by the movement of suture 728 to the left on the page and the arrangement of suture 712 within pleural space 722.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A localization system comprising:
a delivery needle having a proximal end and a distal end;
a localization structure housed within the delivery needle and extending along a length, the localization structure comprising:
a coil portion configured to be arranged within a lung, and
a suture portion configured to be arranged in a pleural space; and
a pusher rod configured to fit into the delivery needle and permit retraction of the delivery needle without corresponding retraction of the localization structure.

2. The localization system of claim 1, wherein the localization structure has a length of between 8 and 10 cm.

3. The localization system of claim 1, wherein the coil is configured to be affixed to a nodule.

4. A method for localizing a nodule of a patient, the method comprising:
locating the nodule;
inserting a preloaded delivery needle from an insertion site to the nodule, wherein the preloaded delivery needle contains a localization structure configured to form a coil affixed to a suture;
inserting a pusher wire into the preloaded delivery needle while retracting the needle to deploy the localization coil within the patient and attached to the nodule;
inserting the pusher wire into the preloaded delivery needle while retracting the needle to deploy the suture through a pleural space to the insertion site;
cutting the suture at the insertion site.

5. The method of claim 4, further comprising creating an incision at the insertion site, and wherein cutting the suture at the insertion site comprises cutting the suture flush with the incision.

6. The method of claim 5, wherein the nodule is located in a lung of the patient.

7. The method of claim 6, wherein the insertion site passes through a pleural space separating the lung from the incision.

8. The method of claim 7, further comprising causing relative movement of the lung relative to the rest of the patient such that the suture is retracted into the pleural space.

9. The method of claim 8, further comprising removing the nodule from the lung.

10. The method of claim 9, wherein the method comprises:
a preoperation phase that includes locating the nodule, inserting the preloaded delivery needle, inserting the pusher wire, and cutting the suture at the insertion site; and
an operation phase that includes locating the suture in the pleural space and removing the nodule.

11. The method of claim 10, wherein the preoperation phase further includes locating the nodule.

12. The method of claim 11, wherein locating the nodule comprises at least one of x-ray, ultrasound, and tomography.

13. A kit comprising:
a delivery needle having a proximal end and a distal end;
a localization structure comprising a coil coupled to a suture; and
a pusher rod configured to fit within the delivery needle.

14. The kit of claim 13, wherein the localization structure is configured to be housed within the delivery needle and extend along a length.

15. The kit of claim 13, wherein the coil is configured to be arranged within a lung and affixed to a nodule.

16. The kit of claim 13, wherein the pusher rod is configured to fit into the delivery needle and permit retraction of the delivery needle without corresponding retraction of the localization structure.

17. The kit of claim 13, wherein the localization structure and the pusher rod are arranged within the delivery needle, with the coil arranged closest the distal end, the suture arranged proximate from the coil, and the pusher rod arranged proximate from the suture.

18. The kit of claim 13, further comprising a cutting implement configured to cut the suture to a desired length.
